(19) United States
(12) Patent Application Publication
(10) Pub. No.: US 2023/0384574 A1
(43) Pub. Date: Nov. 30, 2023

(54) CELL IMAGING SYSTEMS AND METHODS

(71) Applicant: Georgia Tech Research Corporation, Atlanta, GA (US)

(72) Inventors: Shu Jia, Atlanta, GA (US); Jeonghwan Son, Atlanta, GA (US)

(21) Appl. No.: 18/249,423

(22) PCT Filed: Oct. 28, 2021
(86) PCT No.: PCT/US2021/057039

§ 371 Date: Apr. 18, 2023

Related U.S. Application Data
(60) Provisional application No. 63/106,494, filed on Oct. 28, 2020.

(51) Int. Cl. G02B 21/18; C12M 1/32; C12M 1/34; H04N 23/90; G02B 21/16; G02B 21/36; G02B 21/33; G02B 21/02; G02B 21/08

(52) U.S. Cl. CPC G02B 21/18; C12M 23/12; C12M 41/36; H04N 23/90; G02B 21/16; G02B 21/36; G02B 21/33; G02B 21/02; G02B 21/086; H04N 23/56

(57) ABSTRACT

Disclosed herein are prosthesis simulator devices comprising a first restraint configured to restrain one or more fingers of a wearer of the simulator, a second restraint configured to restrain a thumb of the wearer, and a plurality of artificial digits configured to move in a manner to simulate one or more prosthetic fingers and a prosthetic thumb of a prosthesis. The first restraint can be attached to a roof plate connected to a base plate and defining a dorsal side of the prosthesis simulator. The second restraint can be attached to a holster connected to the base plate on a palmar side of the prosthesis simulator. Also disclosed herein are methods of using the same.

### CROSS-REFERENCE TO RELATED APPLICATIONS

This application claims the benefit of U.S. Provisional Application Ser. No. 63/106,494, filed on 28 Oct. 2020, the entire contents and substance of which is incorporated herein by reference in its entirety as if fully set forth below.

### FIELD OF THE DISCLOSURE

The present disclosure relates generally to cell imaging systems and methods.

Particularly, embodiments of the present disclosure relate to miniaturized microscopy for parallel cell imaging, and methods of using the same.

### BACKGROUND

Visualizing diverse anatomical and functional behaviors of single cells provides critical insights into the fundamental principals of living organisms. Conventional assays rely on standard cell fixation to observe and characterize cells at discrete time points. Such methods are insufficient to reveal many dynamic, rare, and heterogeneous cellular events.

The advent of live cell fluorescence imaging has afforded new alternative ways to mediate the limitation of traditional fixed-endpoint imaging. The implement of multi-well plates on fluorescence imaging has improved experimental throughput and enabled the observation of the multiplexed in vitro cell culture conditions and the seeding of different cell types on a singular plate assay for subpopulation analysis. However, conventional systems still use a mechanical serial scanning method with a single objective lens which inherently limits the finite data throughput rate set by the camera and scanning speed. Such methods are limiting, even with the developing quick speed of mechanical actuations. In addition, the systems often require special functions to correct focal drifts caused by mechanical scanning Importantly, incoherent acquisition time between well-to-well images by serial scanning still limits the ability of the conventional systems to achieve synchronized parallel imaging on a multi-well plate.

What is needed, therefore, are prosthesis simulator devices and methods to increase prothesis use and training abilities. Embodiments of the present disclosure address this need as well as other needs that will become apparent upon reading the description below in conjunction with the drawings.

### BRIEF SUMMARY OF THE DISCLOSURE

The present disclosure relates generally to cell imaging systems and methods.

Particularly, embodiments of the present disclosure relate to miniaturized microscopy for parallel cell imaging, and methods of using the same.

An exemplary embodiment of the present disclosure can provide a cell imaging system, comprising: a microscope array comprising a plurality of microscopes; a well plate disposed underneath the microscope array and comprising a plurality of wells, each of the plurality of microscopes corresponding to a respective well of the plurality of wells; and an illumination array underneath the well plate configured to illuminate each of the plurality of wells.

In any of the embodiments disclosed herein, each of the plurality of wells can be configured to contain cells to be imaged.

In any of the embodiments disclosed herein, the cells to be imaged can be in situ cells or fixed status cells.

In any of the embodiments disclosed herein, the illumination array can comprise a plurality of fluorescent light emitters.

In any of the embodiments disclosed herein, the cell imaging system can further comprise: a processor in communication with each of the plurality of microscopes; and a memory storing instructions causing the plurality of microscopes to image each of the plurality of wells.

In any of the embodiments disclosed herein, the memory can further cause the processor to adjust a distance between the microscope array and the well plate such that the microscope array can image a focal plane in each of the plurality of wells.

In any of the embodiments disclosed herein, the memory can cause the plurality of microscopes to synchronously image the plurality of wells.

In any of the embodiments disclosed herein, the memory can cause the plurality of microscopes to image the plurality of wells for a time period of 10 minutes or greater.

In any of the embodiments disclosed herein, the time period can be 60 minutes or greater.

In any of the embodiments disclosed herein, the cell imaging system can further comprise a well plate lid disposed between the well plate and the microscope array.

In any of the embodiments disclosed herein, the well plate lid can comprise threads on an attachment point wherein the microscope array attaches thereto, such that each of the plurality of microscopes can move in a vertical direction orthogonal to the well plate lid.

In any of the embodiments disclosed herein, the plurality of microscopes can each have a lateral resolution of 3 μm or less and an axial resolution of 40 μm or less.

Another example of the present disclosure can provide a cell incubator comprising the system of any of the embodiments disclosed herein.

Another example of the present disclosure can provide a cell imaging system, comprising: a processor in communication with a microscope array; and a memory storing instructions causing the microscope to image a well plate comprising a plurality of cells.

In any of the embodiments disclosed herein, the plurality of cells can comprise in situ cells.

In any of the embodiments disclosed herein, the microscope array can comprise a plurality of microscopes; the well plate can be disposed underneath the microscope array and comprises a plurality of wells, each of the plurality of microscopes corresponding to a respective well of the plurality of wells; and an illumination array underneath the well plate can be configured to illuminate each of the plurality of wells.

In any of the embodiments disclosed herein, the illumination array can comprise a plurality of fluorescent light emitters.

In any of the embodiments disclosed herein, each of the plurality of wells can comprise cells from the plurality of cells.

In any of the embodiments disclosed herein, the memory can further cause the processor to adjust a distance between the microscope array and the well plate such that the microscope array can image a focal plane in each of the plurality of wells.

In any of the embodiments disclosed herein, the memory can cause the plurality of microscopes to synchronously image the plurality of wells.

In any of the embodiments disclosed herein, the memory can cause the plurality of microscopes to image the plurality of wells for a time period of 10 minutes or greater.

In any of the embodiments disclosed herein, the time period can be 60 minutes or greater.

In any of the embodiments disclosed herein, the cell imaging system can further comprise a well plate lid disposed between the well plate and the microscope array.

In any of the embodiments disclosed herein, the well plate lid can comprise threads on an attachment point wherein the microscope array attaches thereto, such that each of the plurality of microscopes can move in a vertical direction orthogonal to the well plate lid.

In any of the embodiments disclosed herein, the plurality of microscopes can each have a lateral resolution of 3 μm or less and an axial resolution of 40 μm or less.

Another embodiment of the present disclosure can provide a cell incubator comprising the system of any of the embodiments disclosed herein.

These and other aspects of the present disclosure are described in the Detailed Description below and the accompanying figures. Other aspects and features of embodiments of the present disclosure will become apparent to those of ordinary skill in the art upon reviewing the following description of specific, exemplary embodiments of the present invention in concert with the figures. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments of the invention discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The accompanying drawings, which are incorporated in and constitute a part of this specification, illustrate multiple embodiments of the presently disclosed subject matter and serve to explain the principles of the presently disclosed subject matter. The drawings are not intended to limit the scope of the presently disclosed subject matter in any manner.

FIG. 1 illustrates a cell imaging system in accordance with the present disclosure.

FIG. 2 illustrates a system diagram of a cell imaging system in accordance with the present disclosure.

FIG. 3 illustrates a component diagram of a computing device in accordance with the present disclosure.

FIG. 4 illustrates a flowchart of a method for imaging cells in accordance with the present disclosure.

FIG. 5 illustrates a cell imaging system in accordance with the present disclosure.

FIG. 6 illustrates a cell imaging system in accordance with the present disclosure.

### DETAILED DESCRIPTION

As described above, the emergence of portable cell imaging strategies has thus far transformed many conventional schemes with high adaptability, cost-effective functionalities, and easy accessibility to cell-based assays. Integrating advanced fabrication, computation, and sensor technologies, these platforms have been reported in various implementations such as lens-free techniques, smartphone imaging, and miniaturized microscopy. Amongst these advancements, for example, lens-free and smartphone imaging methods bypass the restrictions of traditional optical hardware, thereby effectively overcoming either the fundamental trade-offs or the instrumentation complexity of lens-based imaging systems. These paradigmatic breakthroughs have harnessed versatile levels of adaptations and integration for cell diagnosis and analysis.

In contrast, miniaturized microscopy retains commonly adopted imaging features and sample assays of conventional microscopy, while being particularly optimized for time-lapse cell observation in situ. Specifically, various fluorescence modules can be exploited for miniaturized microscopy to investigate cells with high sensitivity and molecular specificity. Such architectures can mainly contain specialized cell chambers, inverted microscope configurations, and mechanical scanning across a larger field of view. However, these features become restrictive for experimental demands such as compatibility with conventional cell culture workflow, accessibility to up-right physiological imaging, integration with biochemical sensors under the cell platform, and parallelization of data acquisition. Furthermore, many of these fluorescence components are implemented as additional modules to the existing bright-field miniaturized systems, adopting a broad-beam illumination or compound objective lenses, therefore providing a less-than-optimal image quality, fluorescence efficiency, and phototoxicity for time-lapse observation. In this context, the fluorescence imaging capability of in situ microscopy remains to be fully utilized to meet ever-increasing live-cell imaging needs.

Disclosed herein is miniaturized modular-array microscopy (MAM) for compact portable fluorescence live-cell imaging in flexible formats. The MAM system is formulated on the basis of the emerging miniscopy technology for functional brain imaging, which has offered remarkable advantages in the fluorescence imaging capability, high flexibility and scalability, and open accessibility to mass-fabricated micro-optics and semiconductor optoelectronics. In the present disclosure, the disclosed systems and methods can utilize miniscopy architecture and the designed compact up-right modular microscopes, implemented with gradient-index (GRIN) objectives and individually addressed illumination and digital modules. The system can provide improved fluorescence efficiency and photo-toxicity, and the architecture can enable parallel data acquisition in situ using conventional off-the-shelf cell chambers. Compared with existing methods, the disclosed modular systems can offer a high optical sensitivity and spatiotemporal resolution (˜3 μm and up to 60 Hz), a configuration compatible with conventional cell culture assays and physiological imaging, and an enhanced imaging ability through parallelization of data acquisition. The system can be demonstrated using various caliber and biological samples and experimental conditions, representing a promising solution to time-lapse in situ single-cell imaging and analysis.

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

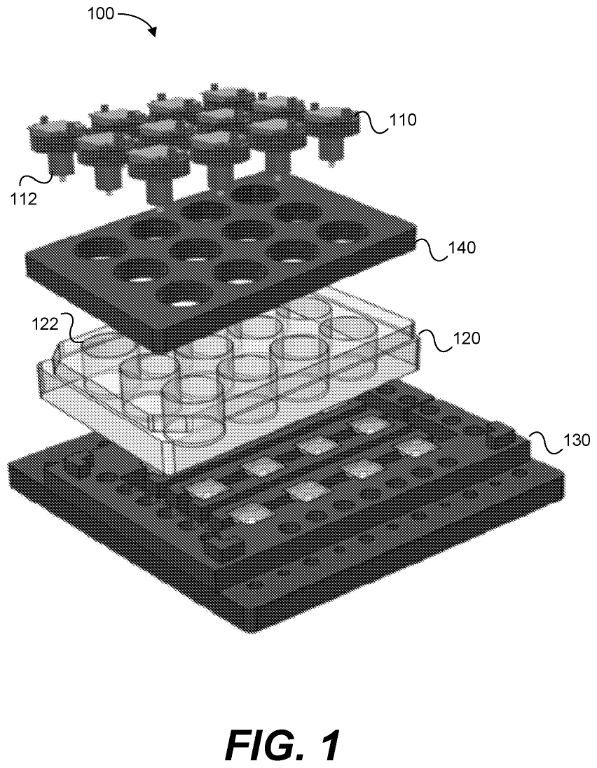

FIG. 1 illustrates a cell imaging system 100. As shown, the cell imaging system 100 can comprise a microscope array 110, a well plate 120, and an illumination array 130. The well plate 120 can be disposed underneath the microscope array 110. Further, the illumination array 130 can be underneath the well plate 120.

The microscope array 110 can comprise a plurality of microscopes, such as the first microscope 112. Further, the well plate 120 can comprise a plurality of wells, such as the first well 122. Each of the plurality of wells can correspond to each of the plurality of microscopes. For example, the first well 122 can be positioned to correspond to the first microscope 112. Additionally, the illumination array 130 can be configured to illuminate each of the plurality of wells.

Each microscope in the microscope array 110 can be uniform. In other words, the microscope array 110 can comprise a plurality of homogeneous microscopes. Alternatively, the microscope array 110 can comprise a plurality of unique and dissimilar microscopes. Each microscope in the microscope array 110 can have a lateral resolution and an axial resolution. The lateral resolution can be 3 μm or less (e.g., 3.5 μm or less, 3 μm or less, 2.5 μm or less, 2 μm or less, 1.5 μm or less, or 1 μm or less). The lateral resolution can also be from 1 μm to 5 μm (e.g., from 1.5 μm to 4.5 μm, from 2 μm to 4 μm, or from 2.5 μm to 3.5 μm). The axial resolution can be 40 μm or less (e.g., 39 μm or less, 38 μm or less, 37 μm or less, 36 μm or less, 35 μm or less, 34 μm or less, 33 μm or less, 32 μm or less, or 31 μm or less). The axial resolution can also be from 30 μm to 50 μm (e.g., from 31 μm to 49 μm, from 32 μm to 48 μm, from 33 μm to 47 μm, from 34 μm to 46 μm, from 35 μm to 45 μm, from 36 μm to 44 μm, from 37 μm to 43 μm, from 38 μm to 42 μm, or from 39 μm to 41 μm).

Each of the plurality of wells can contain cells to be imaged. The cells to be imaged can include in situ cells or fixed status cells. In such a manner, the cells can be static such that a single image can capture the cells. Alternatively, the cells can be in situ, or dynamic, such that the microscope array 110 can continuously image the cells over time. The microscope array 110 can image the plurality of wells synchronously. During the imaging, the illumination array can illuminate the well plate 120 with fluorescent light.

The microscope array 110 can image the well plate for a time period of 10 minutes or greater (e.g., 15 minutes or greater, 20 minutes or greater, 25 minutes or greater, 30 minutes or greater, 35 minutes or greater, 40 minutes or greater, 45 minutes or greater, 50 minutes or greater, 55 minutes or greater, or 60 minutes or greater). In such a manner, the microscope array 110 can continuously image the well plate to monitor the status of the cells in the well plate.

The illumination array 130 can include a plurality of fluorescent light emitters. For example, the illumination array 130 can include a light emitting diode (LED) array. For example, each LED in the illumination array 130 can comprise a 3-color LED (e.g., red, green, and blue). In such a manner, each LED in the illumination array 130 can be illuminated by a different color or combination of colors, thereby creating a multiplexed color scheme. The color scheme of the illumination array can be altered and changed as desired manually, or the color scheme can be controlled automatically by the cell imaging system 100. Each fluorescent light emitter can be positioned under and correspond to each well in the well plate 120. The illumination array 130 can further include lenses and emission filters for each fluorescent light emitter. For example, the illumination array 130 can include a half-ball lens to collimate a diverging beam from each of the fluorescent light emitters.

Furthermore, the cell imaging system 100 can include a well plate lid 140 disposed between the well plate 120 and the microscope array 110. In such a manner, the well plate lid 140 can facilitate attachment between the well plate 120 and the microscope array 110, as well as provide protection for each. The well plate lid 140 can have threads on an attachment point to which the microscope array 110 attaches. In such a manner, by articulating each microscope in the microscope array 110 within the well plate lid 140, the threads can cause each of the microscopes to move in a vertical direction. In other words, due to the threads, the microscope array 110 can move in a direction orthogonal to the well plate lid 140. The microscope array 110 can be moved in the direction orthogonal to the well plate lid 140 manually (e.g., by a user) or automatically (e.g., by the cell imaging system 100, as described below).

Alternatively, the cell imaging system 100 can be configured for inverted microscopy. The microscope array 110 can be positioned as a base, and the well plate 120 can be disposed on top of the microscope array 110. Further, the illumination array 130 can be disposed on top of the well plate 120, thereby sandwiching the well plate 120 between the illumination array 130 and the microscope array 110.

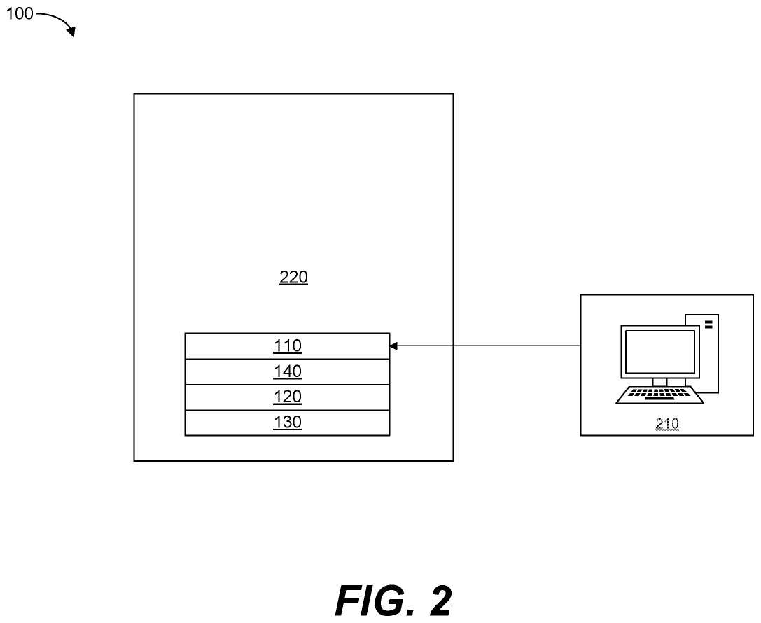

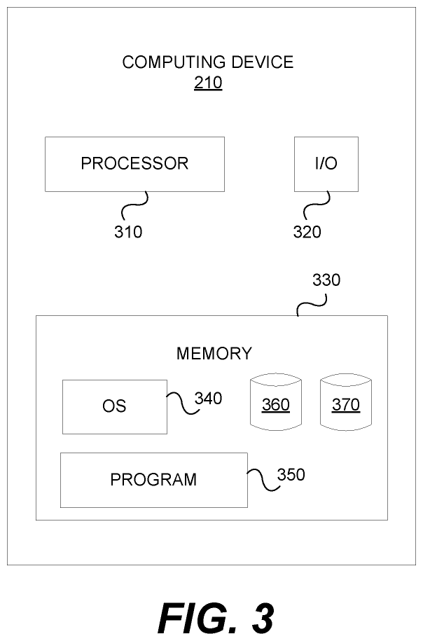

FIG. 2 illustrates a system diagram of the cell imaging system 100. As shown, the cell imaging system 100 can be in communication with a computing device 210. The computing device 210 can be configured to send and/or receive data with the cell imaging system 100. The computing device 210 is described in detail below in FIG. 3. The computing device 210 can further be in communication with specific components of the cell imaging system. For example, the computing device 210 can collect data from the microscope array 110 and articulate the microscope array 110 to move vertically along the threads in the well plate lid 140. Alternatively, or in addition, the microscope array 110 can be moved vertically by manual adjustment. The computing device 210 can also operate the illumination array 130 to turn the fluorescent light emitters on and off and/or adjust the intensity and emission of the illumination array 130.

Furthermore, the cell imaging system 100 can be contained within an incubator 220. The incubator 220 can be used to cultivating and growing cells. In such a manner, cells placed in the well plate 120 can continue to cultivate in the incubator 220 while being imaged by the microscope array 110.

FIG. 3 illustrates an example of the computing device 210. As shown, the computing device 210 can include a processor 310; an input/output (I/O) device 320; a memory 330, which can contain an operating system (OS) 340; a storage device 360, which can be any suitable repository of data; and a program 350. In some examples, the computing device 210 can also include a communication interface, such as a transceiver. In some examples, the computing device 210 can further include a peripheral interface, a mobile network interface in communication with the processor 310, a bus configured to facilitate communication between the various components of the computing device 210, and/or a power source configured to power one or more components of the computing device 210. In certain examples, the computing device 120 can include a geographic location sensor (GLS) for determining the geographic location of the computing device 210.

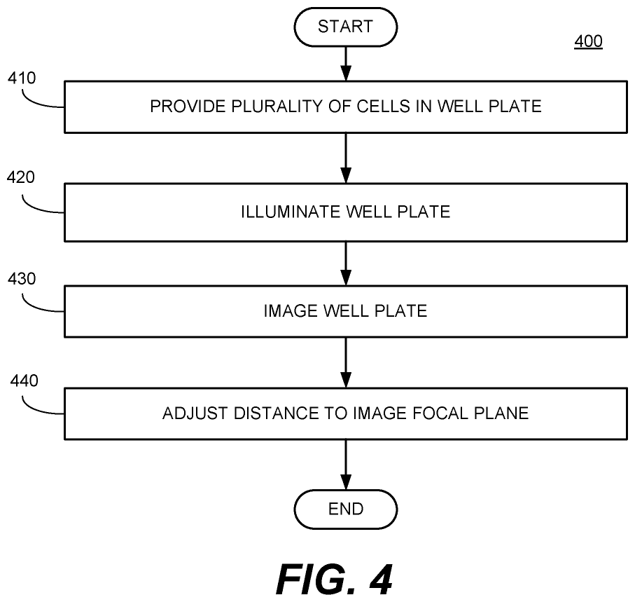

FIG. 4 illustrates a flowchart of a method 400 for imaging cells. While the present method is described with respect to the cell imaging system 100, it is understood that the method 400 can be implemented by other systems for cell imaging.

In block 410, the cell imaging system 100 can provide a plurality of cells contained in the well plate 120. Each of the plurality of wells can contain cells to be imaged by the cell imaging system 100. The cells to be imaged can include in situ cells or fixed status cells. In such a manner, the cells can be static such that a single image can capture the cells. Alternatively, the cells can be in situ, or dynamic, such that the cell imaging system 100 can continuously image the cells over time. The method 400 can then proceed on to block 420.

In block 420, the cell imaging system 100 can illuminate the well plate 120 using the illumination array 130. The illumination array 130 can include a plurality of fluorescent light emitters. For example, the illumination array 130 can include a light emitting diode (LED) array. Each fluorescent light emitter can be positioned under and correspond to each well in the well plate 120. The illumination array 130 can further include lenses and emission filters for each fluorescent light emitter. For example, the illumination array 130 can include a half-ball lens to collimate a diverging beam from each of the fluorescent light emitters. The method 400 can then proceed on to block 430.

In block 430, the cell imaging system 100 can image the well plate 120 and the plurality of cells using the microscope array 110. The microscope array 110 can image the plurality of wells synchronously. The microscope array 110 can comprise a plurality of microscopes, such as the first microscope 112. Further, the well plate 120 can comprise a plurality of wells, such as the first well 122. Each of the plurality of wells can correspond to each of the plurality of microscopes. For example, the first well 122 can be positioned to correspond to the first microscope 112. The method 400 can then proceed on to block 440.

In block 440, the cell imaging system 100 can adjust a distance between the microscope array 110 and the well plate 120. The distance can be a vertical distance in a direction orthogonal to the well plate 120. In such a manner, the microscope array 110 can image a focal plane in the well plate 120, and therefore in each of the plurality of wells. The cell imaging system 100 can include a well plate lid 140 disposed between the well plate 120 and the microscope array 110. In such a manner, the well plate lid 140 can facilitate attachment between the well plate 120 and the microscope array 110, as well as provide protection for each. The well plate lid 140 can have threads on an attachment point to which the microscope array 110 attaches. In such a manner, by articulating each microscope in the microscope array 110 within the well plate lid 140, the threads can cause each of the microscopes to move in a vertical direction. In other words, due to the threads, the microscope array can move in a direction orthogonal to the well plate lid 140. The method 400 can terminate after block 440 or proceed on to other method steps not shown.

As used in this application, the terms “component,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

While the present disclosure has been described in connection with a plurality of exemplary aspects, as illustrated in the various figures and discussed above, it is understood that other similar aspects can be used, or modifications and additions can be made to the described aspects for performing the same function of the present disclosure without deviating therefrom. For example, in various aspects of the disclosure, methods and compositions were described according to aspects of the presently disclosed subject matter. However, other equivalent methods or composition to these described aspects are also contemplated by the teachings herein. Therefore, the present disclosure should not be limited to any single aspect, but rather construed in breadth and scope in accordance with the appended claims.

### Examples

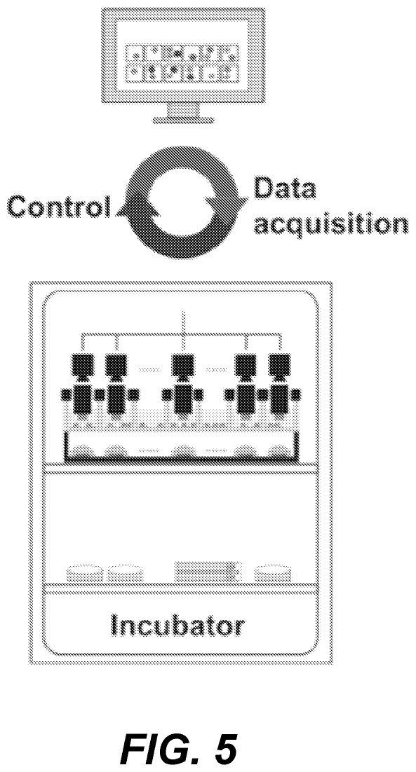

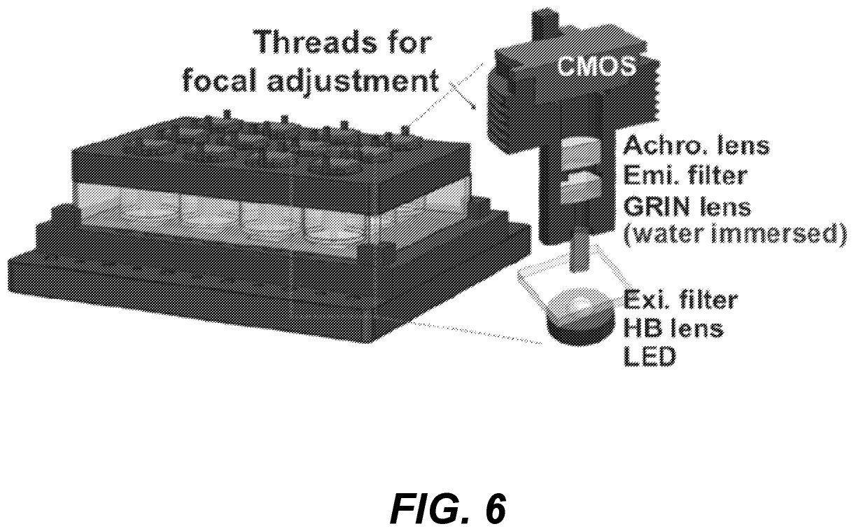

The overall MAM system is designed and fabricated by a 3D resin printer to fit with conventional well plates and a standard incubator (FIG. 5). The fully assembled MAM system is a handheld system in 15×15×3 cm³ size. The array of 12 microscopy units is directly placed on top of a 12 well place with a well plate lid to provide medium-immersed upright configuration. The array of MAM units implies a ‘plug and play’ approachability by scaling different numbers of MANI units. The individual well on a conventional microwell plate is illuminated from the bottom by an excitation panel of a multicolour-LED array that provides 470, 520, and 625 nm excitations. (FIG. 6). Individual MAM units visualize the relative wells but are simultaneously operable to provide the temporally synchronized cell imaging through CMOS sensors. The in-situ cell imaging can be achieved by operating the MAM system inside an incubator and communicating with an external PC for a control and data acquisition.

We measured the optical capability of the MAM system with various testing targets and showed the resolution (laterally <3.0.tm, axially <40.tm) and the effective field of view (300.tm×300.tm). The high portability of the MAM system enables to operate live cell visualization of growing on a microwell plate inside a commercialized incubator in normal cell culture condition. To demonstrate in vitro fluorescent cell imaging on a microtiter plate, we prepared nuclear-stained fixed COS-7 cells on a 12 well plate and imaged sequentially after all MAM units positioned in focal planes. Under the MAM system, the nucleuses were clearly resolved and segmented using the recent open-source nucleus segmentation algorithm in ImageJ.

Inside an incubator, the nucleus of live COS-7 cells was visualized by the MAM system and analyzed to track cellular movement and proliferation. We noticed live cell imaging inside an incubator showed less fluorescent signal to noise (SNR). In order to facilitate cellular analysis with open-source software that are optimized for less-background images, we processed the acquired image stacks by CMOS noise correction to recover SNR. The trajectory of single cell movements over 12 minutes that acquired as 2 frame rates were analyzed then averaged every 10 seconds. The single cell tracking by MAM system also enabled to identify proliferation events of live cells elapsed time of 55 minutes. As results, the MAM system proved the performance of in situ cell imaging in a conventional incubator and continuous tracking of cellular changes in morphology, motility, and proliferation.

1. A cell imaging system comprising:
a first microscope;
a first well configured to contain one or more cells to be imaged;
a first illuminator configured to illuminate the first well;
a processing assembly comprising at least one processor in communication with the first microscope; and
a memory assembly comprising at least one memory storing instructions causing the first microscope to image the first well.

2. (canceled)

3. The cell imaging system of claim 1, wherein one or more of the cells to be imaged is selected from the group consisting of an in situ cell, a fixed status cell, and a combination thereof.

4. The cell imaging system of claim 3, wherein the first illuminator comprises a fluorescent light emitter.

5. The cell imaging system of claim 1 further comprising:
a microscope array comprising the first microscope and one or more additional microscopes;
a well plate comprising the first well and one or more additional wells, the first well associated with the first microscope and at least one of the additional wells associated with a respective microscope of the additional microscopes; and
an illumination array comprising the first illuminator and one or more additional illuminators;
wherein the processing assembly is in communication with at least a portion of the microscopes; and
wherein the memory assembly stores instructions causing at least a portion of the microscopes to image respective wells.

6. The cell imaging system of claim 5, wherein the memory assembly further causes the processing assembly to adjust a distance between the microscope array and the well plate such that the microscope array can image a focal plane in at least a portion of the wells.

7. The cell imaging system of claim 6, wherein the memory assembly further causes two or more of the microscopes to synchronously image respective wells associated with each of the two or more microscopes.

8. The cell imaging system of claim 7, wherein the memory assembly further causes at least one of the microscopes to image a respective well associated with each of the at least one microscopes for a time period of 10 minutes or greater.

9. The cell imaging system of claim 8, wherein the time period is 60 minutes or greater.

10. The cell imaging system of claim 5 further comprising a well plate lid disposed between the well plate and the microscope array;
wherein the well plate lid comprises threads on an attachment point at which point the microscope array attaches such that each of the microscopes can move in a vertical direction orthogonal to the well plate lid.

11. (canceled)

12. The cell imaging system of claim 8, wherein each of the microscopes has a lateral resolution of 3 μm or less and an axial resolution of 40 μm or less.

13. A cell incubator comprising:
the cell imaging system of claim 5.

14.-26. (canceled)

27. The cell imaging system of claim 1 further comprising:
a microscope array comprising the first microscope and one or more additional microscopes;
a well plate comprising the first well and one or more additional wells, the first well associated with the first microscope and each of the additional wells associated with a respective microscope of the additional microscopes;
a well plate lid disposed between the microscope array and the well plate; and
an illumination array comprising the first illuminator and one or more additional illuminators, the first illuminator associated with the first microscope and first well plate, and each of the additional illuminator associated with a respective microscope of the additional microscopes and a respective well of the additional wells; and
wherein the processing assembly is in communication with each of the microscopes; and
wherein the memory assembly stores instructions causing each of the microscopes to image each respective well.

28. The cell imaging system of claim 27, wherein each microscope is configured for liquid immersion.

29. The cell imaging system of claim 28, wherein each microscope is further configured for up-right imaging of one or more cells to be imaged contained in the wells of the well plate.

30. The cell imaging system of claim 27, wherein each microscope comprises a gradient-index (GRIN) objective.

31. The cell imaging system of claim 30, wherein each microscope further comprises:
a dual-band emission filter; and
an achromatic tube lens.

32. The cell imaging system of claim 27, wherein each microscope of the microscope array is configured for individual addressing such that the distribution of the microscopes in the microscope array is customizable into various patterns.

33. The cell imaging system of claim 27, wherein each illuminator comprises a multi-color LED.

34. A cell imaging system, comprising:
a microscope array comprising microscopes having a gradient-index (GRIN) objective and configured for liquid immersion;
a well plate disposed underneath the microscope array and comprising wells configured to contain one or more cells in a liquid for imaging; and
an illumination array underneath the well plate configured to illuminate the wells for cell imaging;
wherein each microscope corresponds to a respective well of the well plate.

35. The cell imaging system of claim 34, wherein the well plate lid comprises threads on an attachment point at which point the microscope array attaches such that each of the microscopes can move in a vertical direction orthogonal to the well plate lid.
